(19) United States
(12) Patent Application Publication
(10) Pub. No.: US 2010/0280292 A1
(43) Pub. Date: Nov. 4, 2010

(54) METHOD OF RECOVERING HYDROFLUORIC ACID

(76) Inventor: Bertrand Collier, Saint-Genis-Laval (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/305,875

(22) PCT Filed: May 21, 2007
(86) PCT No.: PCT/FR07/51303

§ 371 Date: Jul. 15, 2010

(30) Foreign Application Priority Data
Jun. 21, 2006 (FR) 0605524

(51) Int. Cl. C07C 19/08

(52) U.S. Cl. 570/169; 570/165

(57) ABSTRACT

The present invention relates to a process for producing pentafluoroethane. More particularly, the subject of the invention is a continuous process for producing pentafluoroethane comprising (i) a step of fluorinating perchloroethylene (PER) with hydrofluoric acid, in the gas phase, in the presence of a catalyst, (ii) a step of separating the products issuing from step (i) in order to give a fraction of light products and a fraction of heavy products, comprising hydrofluoric acid, unreacted perchloroethylene and under-fluorinated products, and (iii) a step of pretreating the fraction of heavy products before recycling to step (i).

The present invention relates to a method of treating a stream comprising hydrofluoric acid. More particularly, the subject of the invention is a method of recovering hydrofluoric acid.

Due to its ability to dissolve oxides, hydrofluoric acid is an important reactant in aluminum and uranium purification processes. It is also used to attack glass, and as a catalyst for isobutane and butene alkylation reactions in oil refining.

In electroplating factories, it is used in certain surface processes, metal oxide stripping processes and electrochemical polishing processes and in various types of electroplating.

Hydrofluoric acid is also used in the synthesis of many organic compounds containing fluorine, among which is tetrafluoroethylene, and the gases used in refrigeration such as hydrofluorocarbons.

The present invention provides a method of recovering hydrofluoric acid, comprising at least one compression step.

The method according to the present invention is characterized in that it comprises at least one step of compressing a gas stream comprising hydrofluoric acid, the temperature of said stream before the compression step being between −20 and 100° C.

The content of hydrofluoric acid present in the stream subjected to the compression step is preferably between 10% and 90% by weight.

In addition to the hydrofluoric acid, the stream may comprise hydrochloric acid and/or organic compounds such as, for example, pentafluoroethane, tetrafluoroethane, dichlorotrifluoroethane, chlorotetrafluoroethane, difluoromethane, perchloroethylene, dichloromethane, chlorofluoromethane and chlorotrifluoroethane.

The gas stream comprising hydrofluoric acid may come from a liquid-phase or gas-phase fluorination reactor.

When the stream to be treated comes from a high-temperature gas-phase fluorination reactor, the stream is, prior to the compression step, cooled to a temperature of between −20 and 100° C.

At the end of the compression step(s), the pressure of the gas stream is preferably between 5 and 20 bar absolute.

Surprisingly, the presence of hydrofluoric acid in a gas stream limits the rise in the temperature of said stream related to the compression. This limitation has the advantage of reducing the number of intermediate cooling steps between the compression steps.

After the compression step(s), the stream is subjected to at least one separation step in order to recover the hydrofluoric acid.

The separation step consists, in general, of one or more distillation(s) at a pressure of preferably between 5 and 20 bar absolute and at a temperature at the bottom of the distillation column of preferably between 50 and 150° C.

According to one embodiment of the invention, the gas stream from a fluorination reactor and comprising hydrofluoric acid is cooled so as to give a liquid phase and a gas phase. The gas phase is subjected to a compression step, without intermediate cooling and with the temperature of the gas phase being, at the end of the compression, between 50 and 150° C., and then sent to a separation step in order to recover the hydrofluoric acid. The liquid phase is pumped to the desired pressure and also subjected, with the compressed gas phase, to the separation step.

1-5. (canceled)

6. A process for producing pentafluoroethane comprising the steps of:
(i) reacting perchloroethylene with hydrofluoric acid (HF) in the gas phase and in the presence of a catalyst;
(ii) separating the products derived from step (i) to yield
a fraction (A) of light products comprising hydrochloric acid and pentafluoroethane, and
a fraction (B) of heavy products comprising unreacted hydrofluoric acid, unreacted perchloroethylene, and at least one compound comprising trichlorodifluoroethane or olefinic compounds; and
(iii) subjecting fraction (B) to a catalytic pretreatment at a temperature ranging from 280 to 400° C. and with an HF/organic compounds molar ratio ranging from 30 to 150, prior to recycling fraction (B) to step (i).

7. The process of claim 6, wherein said olefinic compounds comprise F1111 and/or F1112a.

8. The process of claim 6, wherein said molar ratio ranges from 60 to 130.

9. The process of claim 6, wherein said temperature ranges from 340 to 370° C.

10. The process of claim 6, wherein said fraction (A) further comprises dichlorotrifluoroethane and/or chlorotetrafluoroethane.

11. The process of claim ID, further comprising after separation of said hydrochloric acid and, optionally, after separation of said pentafluoroethane, the step of subjecting said fraction (A) to a fluorination step with hydrofluoric acid, in the gas phase and in the presence of a catalyst.

12. The process of claim 6, further comprising introducing oxygen in steps (i) and (iii).

13. The process of claim 6, wherein said catalyst is a chromium oxide (Cr₂O₃)-based catalyst.

14. The process of claim 13, wherein said catalyst includes another metal in an oxidation state above zero, said metal comprising Ni, Co, Mn, Zn, or mixtures thereof.
